SEQUENCE LISTING (5 sequences)

SEQ ID NO: 1 - DNA, 2681 bp, Homo sapiens
  CDS (484)..(2283)
ctctctggataggaagaaatatagtagaaccctttgaaaatggatattttcacatattttcgttcagatacaaaagctggcagttactgaaataaggacttgaagttccttcctctttttttatgtcttaagagcaggaaataaagagacagctgaaggtgtagccttgaccaactgaaagggaaatcttcatcctctgaaaaaacatatgtgattctcaaaaaacgcatctggaaaattgataaagaagcgattctgtagattctcccagcgctgttgggctctcaattccttctgtgaaggacaacatatggtgatggggaaatcagaagctttgagaccctctacacctggatatgaatcccccttctaatacttaccagaaatgaaggggatactcagggcagagttctgaatctcaaaacactctactctggcaaaggaatgaagttattggagtgatgacaggaacacgggagaacaatgctctgtttgggctggatatttctttggcttgttgcaggagagcgaattaaaggatttaatatttcaggttgttccacaaaaaaactcctttggacatattctacaaggagtgaagaggaatttgtcttattttgtgatttaccagagccacagaaatcacatttctgccacagaaatcgactctcaccaaaacaagtccctgagcacctgcccttcatgggtagtaacgacctatctgatgtccaatggtaccaacaaccttcgaatggagatccattagaggacattaggaaaagctatcctcacatcattcaggacaaatgtacccttcactttttgaccccaggggtgaataattctgggtcatatatttgtagacccaagatgattaagagcccctatgatgtagcctgttgtgtcaagatgattttagaagttaagccccagacaaatgcatcctgtgagtattccgcatcacataagcaagacctacttcttgggagcactggctctatttcttgccccagtctcagctgccaaagtgatgcacaaagtccagcggtaacctggtacaagaatggaaaactcctctctgtggaaaggagcaaccgaatcgtagtggatgaagtttatgactatcaccagggcacatatgtatgtgattacactcagtcggatactgtgagttcgtggacagtcagagctgttgttcaagtgagaaccattgtgggagacactaaactcaaaccagatattctggatcctgtcgaggacacactggaagtagaacttggaaagcctttaactattagctgcaaagcacgatttggctttgaaagggtctttaaccctgtcataaaatggtacatcaaagattctgacctagagtgggaagtctcagtacctgaggcgaaaagtattaaatccactttaaaggatgaaatcattgagcgtaatatcatcttggaaaaagtcactcagcgtgatcttcgcaggaagtttgtttgctttgtccagaactccattggaaacacaacccagtccgtccaactgaaagaaaagagaggagtggtgctcctgtacatcctgcttggcaccatcgggaccctggtggccgtgctggcggcgagtgccctcctctacaggcactggattgaaatagtgctgctgtaccggacctaccagagcaaggatcagacgcttggggataaaaaggattttgatgctttcgtatcctatgcaaaatggagctcttttccaagtgaggccacttcatctctgagtgaagaacacttggccctgagcctatttcctgatgttttagaaaacaaatatggatatagcctgtgtttgcttgaaagagatgtggctccaggaggagtgtatgcagaagacattgtgagcattattaagagaagcagaagaggaatatttatcttgagccccaactatgtcaatggacccagtatctttgaactacaagcagcagtgaatcttgccttggatgatcaaacactgaaactcattttaattaagttctgttacttccaagagccagagtctctacctcatctcgtgaaaaaagctctcagggttttgcccacagttacttggagaggcttaaaatcagttcctcccaattctaggttctgggccaaaatgcgctaccacatgcctgtgaaaaactctcagggattcacgtggaaccagctcagaattacctctaggatttttcagtggaaaggactcagtagaacagaaaccactgggaggagctcccagcctaaggaatggtgaaatgagccctggagccccctccagtccagtccctgggatagagatgttgctggacagaactcacagctctgtgtgtgtgtgttcaggctgataggaaattcaaagagtctcctgccagcaccaagcaagcttgatggacaatggaatgggattgagactgtggtttagagcctttgatttcctggactggacagacggcgagtgaattctctagaccttgggtactttcagtacacaacacccctaagatttcccagtggtccgagcagaatcagaaaatacagctacttctgccttatggctagggaactgtcatgtctaccatgtattgtacatatgactttatgtatacttgcaatcaaataaatattattttattagaaaaaaaaccggaattc

SEQ ID NO: 2 - protein, 599 aa, Homo sapiens
MLCLGWIFLWLVAGERIKGFNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 3 - DNA, 1626 bp, Homo sapiens
  CDS (1)..(1626)
atgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttcctcgagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgtttggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttaa

SEQ ID NO: 4 - protein, 541 aa, Homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 5 - protein, 422 aa, Artificial Sequence
  IL-18 BP-Fc
MRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQGRSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2009/0297517 A1
(43) Pub. Date: Dec. 3, 2009

(54) METHODS FOR TREATING IL-18 MEDIATED DISORDERS

(75) Inventors: John E. SIMS, Seattle, WA (US); Kendall M. MOHLER, Poulsbo, WA (US); Teresa L. BORN, Kenmore, WA (US)

Correspondence Address: IMMUNEX CORPORATION;LAW DEPARTMENT, 1201 AMGEN COURT WEST, SEATTLE, WA, 98119, US

(73) Assignee: IMMUNEX CORPORATION, Seattle, WA (US)

(21) Appl. No.: 12/534,755

(22) Filed: Aug. 3, 2009

Related U.S. Application Data
(63) Continuation of application No. 11/712,654, filed on Feb. 28, 2007.
Application No. 11/712,654 is a continuation of application No. 09/981,421, filed on Jan. 18, 2002, now abandoned.
(60) Provisional application No. 60/241,408, filed on Oct. 18, 2000.

(51) Int. Cl. A61K 39/395; A61K 38/17

(52) U.S. Cl. 424/133.1; 514/12; 424/158.1

(57) ABSTRACT

The invention pertains to methods for treating medical disorders characterized by elevated levels or abnormal expression of IL-18 by administering an IL-18 antagonist, such as soluble IL-18 receptor, a soluble IL-18 binding protein and/or an antibody.

This application is a continuation of U.S. application Ser. No. 11/712,654, filed on Feb. 28, 2007, which is a continuation of U.S. application Ser. No. 09/981,421, filed on Oct. 17, 2001, and claims the benefit of U.S. provisional application No. 60/241,408, filed on Oct. 18, 2000.

### REFERENCE TO THE SEQUENCE LISTING

The present application is being filed along with a Sequence Listing in electronic format. The Sequence Listing is provided as a file entitled Copy_{—}3086-A_SeqListce_filed101701.txt, created Jul. 31, 2009, which is 31 KB in size. The information in the electronic format of the Sequence Listing is incorporated herein by reference in its entirety.

### FIELD OF THE INVENTION

The invention pertains to methods for treating certain diseases and disorders associated with inflammatory and immunoregulatory responses. More particularly, the present invention involves treating diseases characterized by IL-18 production by administering an IL-18 antagonist to an individual afflicted with such a disease.

### BACKGROUND

IL-18, a cytokine produced by activated macrophages and other cells, induces natural killer cell cytotoxicity and participates in the polarization of the T-lymphocyte helper type 1 phenotype. In addition, IL-18 induces interferon-γ (IFNγ) production in antigen-stimulated T-cell lines, and acts synergistically with IL-12 to stimulate IFNγ production in Th1 clones.

Elevated levels of IL-18 have been observed in various disease states including Crohn's disease and rheumatoid arthritis (RA). For example, Pallone and co-workers found that IL-18 was upregulated in mucosal intestinal tissue and lamina propria mononuclear cells from patients with Crohn's disease as compared to samples from patients without an inflammatory bowel disease (Monteleone et al, 1999, J. Immunol. 63: 143-7). Another group which reported similar results in their studies of Crohn's disease specimens, also reported a trend of increased IL-18 expression in colonic surgical specimens from patients with ulcerative colitis (Pizzarro et al, 1999, J. Immunol. 162:6829-35). In studies of RA, McInnes and coworkers reported that IL-18 was expressed in RA synovial membrane, and that administration of recombinant IL-18 can promote erosive collagen-induced arthritis in an animal model (when administered immediately before and during collagen priming and challenge) (Leung et al., 1999, J. Immunol. 164:6495-6502; Gracie et at., 1999, J. Clin. Invest. 104:1393-1401).

These results led the above-cited authors to speculate that IL-18 may play a key pathogenic role in initiating such Th1-mediated disorders. However, another investigator warned that, because of the pleiotrophic roles that IL-18 is likely to play, one should not conclude that blocking IL-18 would help in treating, for example, rheumatoid arthritis (Dayer, 1999, J. Clin. Invest. 104:1337-1339). In addition to its roles in host defense and the suppression of allergies, IL-18 induces nitric oxide (NO) production. Dayer reasoned that induction of NO may be a counter-regulatory loop for IL-18 because NO inhibits the IL-1β-converting enzyme ICE, and thus blocks the processing of proIL-18 into a biologically active cytokine (Id.). By inducing NO, IL-18 decreases its own activity (Id.). Therefore, inhibition of IL-18 could increase ICE activity and promote the maturation of IL-18 and IL-1β, thereby promoting inflammation and tissue destruction (Id.).

Thus, the art showed that it was unclear whether attempts to decrease IL-18 would actually lead to therapeutic results in diseases such as arthritis and inflammatory bowel diseases. Accordingly, there is a need in the art to resolve this dilemma.

### SUMMARY OF THE INVENTION

The invention is based, in part, on the discovery through actual in vivo experimentation that inhibition of IL-18 can indeed be used to treat inflammatory diseases. Therefore, provided herein are methods for treating medical disorders associated with IL-18 mediated inflammatory reactions and/or IL-18 mediated immunoregulatory reactions. The methods of the present invention include administering an IL-18 antagonist that inhibits IL-18 inflammatory and/or immunoregulatory signaling to an individual afflicted with an inflammatory and/or immunoregulatory disease mediated by IL-18. More particularly, the present invention involves administering an IL-18 antagonist such as, for example, a soluble IL-18 receptor, an IL-18 binding protein, and/or an antibody, to such an individual, for a period of time sufficient to induce a sustained improvement in the patient's condition. The invention also provides, in part, the use of an IL-18 antagonist in the manufacture of a medicament for the treatment of medical disorders associated with IL-18 mediated inflammatory reactions and/or IL-18 mediated immunoregulatory reactions.

### BRIEF DESCRIPTION OF THE FIGURES

FIG. 1. Effect of IL-18BP-Fc and M147 Administration on Weight Loss in Mouse Model of Inflammatory Bowel Disease. This figure is a graph of the average % weight change of mice in each treatment group (n=8) as a function of days of treatment with 2% DSS, or no DSS, in the drinking water. Treatment groups were as follows: no DSS, filled squares; 2% DSS+Human IgG control antibody (250 μg/day), open squares; 2% DSS+IL-18BP-Fc fusion protein (600 μg/day), triangles; 2% DSS+M147 antibody (250 μg/day), circles.

FIG. 2. Cytokine Production by MLN Cells After Stimulation With CD3. FIG. 2A illustrates the average level (n=4) of IFNγ production by MLN cells from each treatment group after stimulation by CD3. FIG. 2B illustrates the average level (n=4) of IL-10 production by MLN cells from each treatment group after stimulation by CD3. Treatment groups are indicated and were: no DSS; 2% DSS+Human IgG control antibody (250 μg/day); 2% DSS+IL-18BP-Fc fusion protein (600 μg/day); 2% DSS+M147 antibody (250 μg/day).

FIG. 3. RNase Protection Assays (RPA) Of mRNA Isolated From Large Intestine. The relative levels of mRNAs in the large intestine, as measured by RPA, encoding for IFNγ, TNFα, IL-6, IL-10, IL-18 and IL-IRA are shown in FIG. 3A. The relative levels of mRNAs in the large intestine encoding for IL-1α and IL-1β are shown in FIG. 3B. Treatment groups were as follows: no DSS, purple bars; 2% DSS+Human IgG control antibody (250 μg/day), black bars; 2% DSS+IL-18BP-Fc fusion protein (600 μg/day), blue bars; 2% DSS+M147 antibody (250 μg/day), orange bars.

### DETAILED DESCRIPTION OF THE INVENTION

The present invention provides methods for treating an individual including a human, who is suffering from a medical disorder associated with IL-18 mediated inflammatory reactions or IL-18 mediated immunoregulatory reactions. For purposes of this disclosure, the terms “illness,” “disease,” “medical condition” or “abnormal condition” are used interchangeably with the term “medical disorder.”

Basis, in part, for the invention is the discovery that inhibitors of IL-18 are effective in vivo for treating diseases. Specifically, an IL-18 antagonist fusion protein, IL-18BP-Fc, was found to be useful in preventing experimentally-induced rheumatoid arthritis in a mouse model of this disease. Moreover, the IL-18 antagonist also inhibited the progression of an already established disease in the same animal model. In addition, two different IL-18 antagonists, a viral p13 protein and an IL-18BP-Fc, were also found to be beneficial in ameliorating the deleterious effects of two different animal models of inflammatory bowel diseases. Thus, these in vivo data indicate that inhibition of IL-18 is effective for treating arthritis, rheumatic diseases, and inflammatory gastrointestinal diseases. Any method that neutralizes IL-18 activity or inhibits expression of the IL-18 gene (either transcription or translation) can be used to reduce the inflammatory response caused by IL-18.

The subject methods involve administering to the patient an IL-18 antagonist that is capable of reducing the effective amount of endogenous biologically active IL-18, such as by reducing the amount of IL-18 produced, or by preventing the binding of IL-18 to its cell surface receptor. Such antagonists include receptor-binding peptide fragments of IL-18, IL-18 binding proteins, antibodies directed against IL-18 or a subunit of the IL-18 receptor, inhibitors (e.g., small molecules and peptides) of IL-18 receptor aggregation and signal transduction, and recombinant proteins comprising all or portions of a receptor for IL-18 or modified variants thereof, including genetically-modified muteins, multimeric forms and sustained-release formulations. Particular antagonists include IL-18 binding protein, antagonistic IL-18 receptor antibodies and soluble forms of an IL-18 receptor. Further, suitable IL-18 antagonists encompass chimeric proteins that include portions of both an antibody molecule and an IL-18 antagonist molecule. Such chimeric molecules may form monomers, dimers or higher order multimers. Other suitable IL-18 antagonists include peptides derived from IL-18 that are capable of binding competitively to the IL-18 signaling receptor, yet do not induce signaling, and nucleic acid based antagonists.

In a preferred aspect, protein-based therapeutics can be used to inhibit the activity of IL-18 protein. For example, preferred methods of the invention utilize IL-18 receptor in a form that binds IL-18, and blocks IL-18 signal transduction, thereby interrupting the proinflammatory and immunoregulatory effects of IL-18. PCT Publication WO 99/37772, incorporated in its entirety by reference herein, describes the IL-18 receptor, which is a heterodimeric protein containing an IL-18 binding subunit termed IL-1Rrp1, and an accessory subunit termed AcPL. Although the IL-Rrp1 subunit alone will bind IL-18, its affinity for IL-18 is increased dramatically when present in a heterodimeric complex with the AcPL subunit.

The IL-1Rrp1 polynucleotide sequence and the amino acid sequence that it encodes are provided herein as SEQ ID NO:3 and SEQ ID NO:4, respectively. The soluble extracellular portion of the IL-1Rrp1 subunit that binds IL-18 is represented by amino acids 20 to 329 of SEQ ID NO:4; cleavage of the signal sequence occurs just after amino acid residue 19 of SEQ ID NO:4. However, fragments as small as amino acid residues 20 to 123 and amino acid residues 20 to 226 of SEQ ID NO:4 have been reported to bind IL-18 and can also be used. The IL-1Rrp1 polypeptide is also described in U.S. Pat. No. 5,776,731, incorporated in its entirety by reference herein.

The AcPL polynucleotide sequence and the amino acid sequence that it encodes are provided herein as SEQ ID NO: 1 and SEQ ID NO:2, respectively. The mature extracellular domain of AcPL consists of amino acids 15 to 356 of SEQ ID NO:2; cleavage of the signal sequence occurs just after amino acid residue 14 of SEQ ID NO:2. The AcPL polypeptide, and soluble extracellular fragments thereof, are also described in WO 99/37773, incorporated in its entirety by reference herein. Preferable forms of the IL-18 receptor polypeptides are truncated soluble fragments that retain the capability of binding IL-18. Soluble IL-18 receptor molecules include, for example, analogs or fragments of native IL-18 receptor having at least 20 amino acids, preferably at least 100 amino acids, that lack the transmembrane regions of the native molecule, and that are capable of binding IL-18.

One preferred soluble form of an IL-18 receptor for use in the methods of the present invention includes amino acids 1-329 (20-329 after cleavage of the signal sequence) of SEQ ID NO:4. An even more preferred soluble form of IL-18 receptor is a heterodimeric receptor that includes at least amino acid residues 20-123, 20-226 or 20-329 of SEQ ID NO:4 (the IL-1Rrp1 subunit), and at least amino acids 15-340 of SEQ ID NO:2 (the AcPL subunit), in a covalent or non-covalent association.

Another preferred soluble IL-18 antagonist for use in the methods of the present invention is the IL-18 binding protein. PCT Publication WO 99/09063 describes the IL-18 binding protein, including useful soluble fragments thereof, and this description is incorporated by reference herein. A particularly useful form of the IL-18 binding protein is a fusion with an Fc domain of an antibody. The amino acid sequence of an example of such a fusion protein, termed IL-18BP-Fc herein, is presented in SEQ ID NO:5. This 422 amino acid protein, when expressed in a mammalian cell, will be secreted; the mature secreted form of the protein contains amino acid residues 29-422. Of these residues, amino acid residues 29-192 represent the IL-18 binding protein portion of the molecule, and amino acid residues 193-422 represent the Fc portion of the molecule. The Fc region facilitates purification and dimerization of the fusion polypeptide.

Antagonists derived from IL-18 receptors and IL-18 binding protein (e.g. soluble forms that bind IL-18) compete for IL-18 with IL-18 receptors on the cell surface, thus inhibiting IL-18 from binding to cells, thereby preventing it from manifesting its biological activities. Binding of soluble IL-18 receptor or IL-18 binding protein can be assayed using ELISA or any other convenient assay.

Other types of protein-based therapeutics are antibodies that specifically recognize one or more epitopes of IL-18, or epitopes of conserved variants of IL-18, or peptide fragments of the IL-18 polypeptide that competitively inhibit IL-18 activity. Antibodies to IL-18 can most conveniently be raised to a recombinantly produced form of the protein. For example, human IL-18 has been recombinantly produced from both a cloned cDNA (Ushio et al., 1996, J. Immunol. 156:4274-4279) and cloned genomic DNA (U.S. Pat. No. 6,060,283). Or, antibodies that specifically recognize a component of the IL-18 receptor and that prevent signaling through the receptor by IL-18 can be used to inhibit IL-18 activity. IL-18 antagonists that are antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Thus, such antibodies can, therefore, be utilized as part of inflammatory disorder treatment methods.

For the production of antibodies, various host animals can be immunized by injection with the IL-18 polypeptide, truncated IL-18 polypeptides, a component of the IL-18 receptor (e.g., the IL-18 binding subunit, or the AcPL subunit), a truncated version of a component of the IL-18 receptor, and functional equivalents and mutants thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Alternatively, libraries of antibody fragments can be screened and used to develop human antibodies through recombinant techniques. Such libraries are commercially available from, for example, Cambridge Antibody Technology (Melbourne, UK), and Morphosys (Munich, DE).

Monoclonal antibodies, which are homogeneous populations of antibodies to a particular antigen, can be obtained by any technique that provides for the production of antibody molecules by continuous cell lines in culture. These include, but are not limited to, the hybridoma technique of Kohler and Milstein, (U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb may be cultivated in vitro or in vivo. Or, the antibody genes can be cloned and optionally otherwise altered, and expressed in another cell line approved for recombinant production of protein pharmaceuticals such as, for example, CHO cells.

In addition, techniques developed for the production of “chimeric antibodies” (Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region.

Preferably, for use in humans, the antibodies are human or humanized; techniques for creating such human or humanized antibodies are also well known and are commercially available from, for example, Protein Design Labs, Inc. (Fremont, Calif.), Medarex Inc. (Princeton, N.J.) and Abgennix Inc. (Fremont, Calif.).

Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against IL-18 gene products. Single chain antibodies are formed by linking the heavy and light chain fragments of the Fv region via an amino acid bridge, resulting in a single chain polypeptide.

Antibody fragments which recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the (ab′)₂ fragments. Alternatively, Fab expression libraries can be constructed (Huse et al., 1989, Science, 246:1275-1281) to allow rapid and easy identification of monoclonal Fab fragments with the desired specificity.

Still another IL-18 antagonist that can be used in the methods and compositions of the invention is a virally encoded IL-18 binding protein. For example, the fowlpox (ectromelia) virus p13 polypeptide has been shown to bind to, and inhibit the biological activity, of IL-18 (Born et al., 2000, J. Immunol. 164(6):3246-54, incorporated in its entirety by reference herein). The invention also encompasses the use of IL-18 antagonists yet to be discovered in the therapeutic methods and compositions.

In alternative embodiments, nucleic acid-based immuno therapy can be designed to reduce the level of endogenous IL-18 gene expression, e.g., using antisense or ribozyme approaches to inhibit or prevent translation of IL-18 mRNA transcripts; triple helix approaches to inhibit transcription of the IL-18 gene; or targeted homologous recombination to inactivate or “knock out” the IL-18 gene or its endogenous promoter.

Antisense approaches involve the design of oligonucleotides (either DNA or RNA) that are complementary to IL-18 mRNA. The antisense oligonucleotides will bind to the complementary IL-18 mRNA transcripts and prevent translation. The IL-18 cDNA sequence is described in Ushio et al., 1996, J. Immunol. 156:4274-4279.

Absolute complementarity to the mRNA transcript, although preferred, is not required. A sequence “complementary” to a portion of an RNA, as referred to herein, means a sequence having sufficient complementarity to be able to hybridize with the RNA, forming a stable duplex. In the case of double-stranded antisense nucleic acids, a single strand of the duplex DNA can thus be tested, or triplex formation can be assayed. The ability to hybridize will depend on both the degree of complementarity and the length of the antisense nucleic acid.

Oligonucleotides that are complementary to the 5′ end of the message, e.g., the 5′ untranslated sequence up to and including the AUG initiation codon, should work most efficiently at inhibiting translation. However, oligonucleotides complementary to either the 5′- or 3′-non-translated, and any of the coding and/or non-coding regions of the IL-18 gene transcript could be used in an antisense approach to inhibit translation of endogenous IL-18 mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane such as lipid carriers (see, e.g., Letsinger et al, 1989, Proc. Natl. Acad. Sci. U.S.A. 86:6553-6556; Lemaitre et al, 1987, Proc. Natl. Acad. Sci. 84:648-652; PCT Publication No. WO88/09810, published Dec. 15, 1988), or hybridization-triggered cleavage agents or intercalating agents. (See, e.g., Zon, 1988, Pharm. Res. 5:539-549).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al, 1988, Nucl. Acids Res. 16:3209. Methylphosphonate oligonucleotides can be prepared by use of controlled pore glass polymer supports (Sarin et al, 1988, Proc. Natl. Acad. Sci. U.S.A. 85:7448-7451).

The antisense molecules should be delivered to cells that express the IL-18 transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense sequence is placed under the control of a strong pol III or pol II promoter. The use of such a construct to transfect target cells in the patient will result in the transcription of sufficient amounts of single stranded RNAs that will form complementary base pairs with the endogenous IL-18 gene transcripts and thereby prevent translation of the IL-18 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave IL-18 mRNA transcripts can also be used to prevent translation of IL-18 mRNA and expression of IL-18 protein. (See, e.g., PCT International Publication WO90/11364; U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (International Patent Application No. WO 88/04300; Been and Cech, 1986, Cell 47:207-216).

As in the antisense approach, the ribozymes can be composed of modified nucleotides (e.g. for improved stability, targeting, etc.) and should be delivered to cells which express the IL-18 polypeptide in vivo. A preferred method of delivery involves using a DNA construct “encoding” the ribozyme under the control of a strong constitutive pol III or pol II promoter, so that transfected cells will produce sufficient quantities of the ribozyme to destroy endogenous IL-18 polypeptide messages and inhibit translation. Because ribozymes, unlike antisense molecules, are catalytic, a lower intracellular concentration is required for efficiency.

This invention additionally provides for the use of IL-18 antagonists in the manufacture of a medicament for the treatment of numerous diseases. This invention additionally provides for the use of polynucleotides encoding IL-18 antagonists in the manufacture of IL-18 antagonists for use in the manufacture of a medicament for the treatment of diseases disclosed herein.

Soluble IL-18 antagonists that are polypeptides suitable in the practice of this invention can be fused with a second polypeptide to form a chimeric protein. In one embodiment of such a chimeric protein, the second polypeptide can promote the spontaneous formation by the chimeric protein of a dimer, trimer or higher order multimer that is capable of binding IL-18 molecule and preventing it from binding to a cell-bound receptor that promotes IL-18 signaling. For example, chimeric proteins used as antagonists can be proteins that contain portions of both an antibody molecule and a soluble IL-18 antagonist. In particular aspects, the Fc portion of an antibody molecule can be used. One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al, 1994, EMBO J. 13:3992-4001. Another example of a oligomerization domain is a leucine zipper, the use of which is well known in the art. Indeed, any oligomerization domain known or yet to be discovered can be used as the second polypeptide. One preferred oligomer IL-18 antagonist suitable for treating diseases in humans and other mammals is the IL-18BP-Fc (IL-18 binding protein fused to an Fc mutein region; SEQ ID NO:5) whose use is illustrating below by way of non-limiting working examples. Other preferred oligomer IL-18 antagonists are any of the soluble IL-18 receptor molecules described above fused to either an Fc mutein region or a leuzine zipper or any other oligomerization domain.

In one preferred embodiment of the invention, sustained-release forms of soluble IL-18 antagonists, and in particular, soluble IL-18 receptor or IL-18 binding protein, are used. Sustained-release forms suitable for use in the disclosed methods include, but are not limited to, IL-18 antagonists that are encapsulated in a slowly-dissolving biocompatible polymer, admixed with such a polymer, and or encased in a biocompatible semi-permeable implant. In addition, the IL-18 antagonist can be conjugated with polyethylene glycol (pegylated) to prolong its serum half-life or to enhance protein delivery.

To treat a medical disorder characterized by abnormal or excess expression of IL-18 or abnormal or excess IL-18 signaling, a molecule comprising an IL-18 antagonist, preferably a soluble IL-18 receptor or IL-18 binding protein, or an antibody, is administered to the patient in an amount and for a time sufficient to induce a sustained improvement in at least one indicator that reflects the severity of the disorder. An improvement is considered “sustained” if the patient exhibits the improvement on at least two occasions separated by one to four weeks. The degree of improvement is determined based on signs or symptoms, and may also employ questionnaires that are administered to the patient, such as quality-of-life questionnaires. A therapeutically effective amount of an IL-18 antagonist is that sufficient to achieve such a sustained improvement.

Various indicators that reflect the extent of the patient's illness may be assessed for determining whether the amount and time of the treatment is sufficient. The baseline value for the chosen indicator or indicators is established by examination of the patient prior to administration of the first dose of the soluble IL-18 receptor or other IL-18 antagonist. Preferably, the baseline examination is done within about 60 days of administering the first dose.

Improvement is induced by repeatedly administering a dose of IL-18 antagonist until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating acute conditions.

Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Any efficacious route of administration can be used to therapeutically administer a soluble IL-18 receptor or other IL-18 antagonists. If injected, an IL-18 antagonist can be administered, for example, via intra-articular, intravenous, intramuscular, intralesional, intraperitoneal or subcutaneous routes by bolus injection or by continuous infusion. Other suitable means of administration include sustained release from implants, aerosol inhalation, eyedrops, oral preparations, including pills, syrups, lozenges or chewing gum, topical preparations such as lotions, gels, sprays, ointments, buccal preparations, or other suitable techniques. Alternatively, IL-18 antagonist polypeptides, such as a soluble IL-18 receptor or IL-18 binding protein, can be administered by implanting cultured cells that express the protein; for example, by implanting cells which express a soluble IL-18 receptor or an IL-18 binding protein. In one embodiment, the patient's own cells are induced to produce by transfection in vivo or ex vivo with a polynucleotide that encodes an IL-18 antagonist, and particularly soluble IL-18 receptor or IL-18 binding protein. This polynucleotide can be introduced into the patient's cells, for example, by injecting naked DNA or liposome-encapsulated DNA that encodes soluble IL-18 receptor or other selected IL-18 antagonist, or by other means of transfection. When an IL-18 antagonist is administered in combination with one or more other biologically active compounds, these can be administered by the same or by different routes, and can be administered simultaneously, separately or sequentially.

Soluble IL-18 receptor or IL-18 binding protein or other antagonists of IL-18 preferably are administered in the form of a physiologically acceptable composition comprising purified recombinant protein in conjunction with physiologically acceptable carriers, excipients or diluents. Such carriers are nontoxic to recipients at the dosages and concentrations employed. Ordinarily, preparing such compositions entails combining the IL-18 antagonist with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and other stabilizers and excipients. Neutral buffered saline or saline mixed with conspecific serum albumin are exemplary appropriate diluents. The IL-18 antagonist preferably is formulated as a lyophilizate using appropriate excipient solutions (e.g., sucrose) as diluents. Appropriate dosages can be determined in standard dosing trials, and may vary according to the chosen route of administration. In accordance with appropriate industry standards, preservatives may also be added, such as benzyl alcohol. The amount and frequency of administration will depend, of course, on such factors as the nature and severity of the indication being treated, the desired response, the age and condition of the patient, and so forth.

In one embodiment of the invention, IL-18 antagonist is administered one time per week to treat the various medical disorders disclosed herein, in another embodiment is administered at least two times per week, and in another embodiment is administered at least once per day. An adult patient is a person who is 18 years of age or older. If injected, the effective amount, per adult dose, of an IL-18 binding protein or an IL-18 receptor protein ranges from 1-200 mg/m², or from 1-40 mg/m² or about 5-25 mg/m². Alternatively, a flat dose may be administered, whose amount may range from 2-400 mg/dose, 2-100 mg/dose or from about 10-80 mg/dose. If the dose is to be administered more than one time per week, an exemplary dose range is the same as the foregoing described dose ranges or lower. Preferably, such IL-18 antagonists are administered two or more times per week at a per dose range of 25-100 mg/dose. In one embodiment of the invention, the various indications described below are treated by administering a preparation acceptable for injection containing an IL-18 binding protein at 80-100 mg/dose, or alternatively, containing 80 mg per dose. If the IL-18 antagonist is an antibody, the dose can be from 0.1 to 10 mg/kg, preferably given intravenously as a 15 minute to 3 hour infusion. The dose is administered repeatedly at biweekly, weekly, or separated by several (2-8 weeks).

If a route of administration of IL-18 antagonist other than injection is used, the dose is appropriately adjusted in accord with standard medical practices. For example, if the route of administration is inhalation, dosing may be one to seven times per week at dose ranges from 10 mg/dose to 50 mg per dose.

In many instances, an improvement in a patient's condition will be obtained by injecting a dose of up to about 100 mg of a soluble IL-18 receptor or IL-18 binding protein or an antagonistic antibody one to three times per week over a period of at least three weeks, though treatment for longer periods may be necessary to induce the desired degree of improvement. For incurable chronic conditions, the regimen may be continued indefinitely.

For pediatric patients (age 4-17), a suitable regimen involves the subcutaneous injection of 0.4 mg/kg to 5 mg/kg of IL-18 receptor or IL-18 binding protein, administered by subcutaneous injection one or more times per week.

The invention further includes the administration of IL-18 antagonist concurrently with one or more other drugs that are administered to the same patient, each drug being administered according to a regimen suitable for that medicament. This encompasses pre-treatment, simultaneous treatment, sequential treatment and alternating regimens. Examples of such drugs include but are not limited to antivirals, antibiotics, analgesics, corticosteroids, antagonists of inflammatory cytokines, DMARDs and non-steroidal anti-inflammatories. Additionally, one type of IL-18 antagonist can be combined with a second IL-18 antagonist, including an antibody against IL-18 or against an IL-18 receptor, additional IL-18 receptor derivatives, or other molecules that reduce endogenous IL-18 levels, such as peptidomimetic IL-18 antagonists.

In one preferred embodiment of the invention, the various medical disorders disclosed herein as being treatable with an IL-18 antagonist are treated in combination with another cytokine or cytokine inhibitor. For example, IL-18 antagonist can be administered in a composition that also contains a compound that inhibits the interaction of other inflammatory cytokines with their receptors. The IL-18 antagonist and other cytokine inhibitor can be administered as separate compositions, and these can be administered by the same or different routes. Examples of cytokine inhibitors used in combination with IL-18 antagonist include those that antagonize, for example, IFNγ, IL-6, IL-8, IL-12, IL-15 and TNF, particularly TNFα. Anti-inflamatory cytokines include but are not limited to IL-4, TGFβ, and EGF. Other combinations for treating the hereindescribed diseases include the use of IL-18 antagonist with compounds that interfere with the binding of RANK and RANK-ligand, such as RANK-ligand inhibitors, or soluble forms of RANK, including RANK:Fc. For example, the combination of IL-18 antagonist and RANK:Fc are useful for preventing bone destruction in various settings including but not limited to various rheumatic disorders. Still another combination for treating the hereindescribed diseases include the use of an IL-18 antagonist in combination with an IL-1 antagonist, such as, for example, a soluble IL-1 receptor type II molecule or an antagonistic antibody to the IL-1 receptor. In addition, IL-18 antagonist can be administered in combination with soluble forms of an IL-17 receptor (such as IL-17R:Fc), IL-12 binding protein, or antibodies against CD30-ligand or against CD4.

The present invention further encompasses methods for treating the herein disclosed medical disorders with a combination of an IL-18 antagonist and a TNF inhibitor, preferably TNFR:Fc (ENBREL® marketed by Immunex Corp) and optionally with any combination of the above described cytokines or cytokine inhibitors that are active agents in combination therapies. For example, in accordance with the present invention, combination therapy methods for treating rheumatic, arthritic and various inflammatory gastrointestinal diseases include administering IL-18 antagonist and ENBREL®. Thus, the present invention also relates to the using IL-18 antagonists and TNF inhibitors in combination therapies for use in medicine and in particular in therapeutic and preventive therapies for the medical disorders described herein. The use in medicine can involve the treatment of any of the medical disorders as described herein with a combination therapy that includes administering a combination of an IL-18 antagonist and ENBREL®. The IL-18 antagonists (e.g., soluble IL-18 receptor or IL-18 binding protein or an antagonistic antibody) and TNF inhibitor (e.g., ENBREL®) may be in the form of compounds, compositions or combination therapies. Where the compounds are used together with one or more other components, the compound and the one or more other components can be administered simultaneously, separately or sequentially (usually in pharmaceutical format).

The present invention also relates to the use of IL-18 antagonists (as disclosed), such as, for example, a soluble IL-18 receptor, in the manufacture of a medicament for the prevention or therapeutic treatment of each medical disorder disclosed herein.

Conditions of the gastrointestinal system are treatable or preventable with IL-18 antagonists, compositions or combination therapies, including coeliac disease. For example, IL-18 antagonist compositions, with or without TNF inhibitors (ENBREL®) or other active agents described above are suitable for treating or preventing coeliac disease. In addition, the compounds, compositions and combination therapies of the invention are suitable for treating or preventing Crohn's disease; ulcerative colitis; idiopathic gastroparesis; pancreatitis, including chronic pancreatitis; inflammatory bowel disease and ulcers, including gastric and duodenal ulcers.

Other embodiments of the present invention include methods for using the disclosed IL-18 antagonists, in particular soluble IL-18 receptor, compositions or combination therapies, e.g. soluble IL-18 receptor and ENBREL®, to treat or prevent a variety of rheumatic disorders. These include adult and juvenile rheumatoid arthritis; scleroderma; systemic lupus erythematosus; gout; osteoarthritis; polymyalgia rheumatica; seronegative spondylarthropathies, including ankylosing spondylitis, and Reiter's disease. The subject IL-18 antagonists, compositions and combination therapies are used also to treat psoriatic arthritis and chronic Lyme arthritis. Also treatable or preventable with these compounds, compositions and combination therapies are Still's disease and uveitis associated with rheumatoid arthritis. In addition, the compounds, compositions and combination therapies of the invention are used in treating disorders resulting in inflammation of the voluntary muscle and other muscles, including dermatomyositis, inclusion body myositis, polymyositis, and lymphangioleimyomatosis.

IL-18 antagonist can be used to treat psoriatic arthritis in combination with one, two, three or more other medications that are effective against psoriasis. These additional medications can be administered before, simultaneously with, or sequentially with the soluble IL-18 receptor. Drugs suitable for combination therapies include pain medications (analgesics), including but not limited to acetaminophen, codeine, propoxyphene napsylate, oxycodone hydrochloride, hydrocodone bitartrate and tramadol. In addition, IL-18 antagonist can be administered in combination with a soluble TNF receptor (ENBREL®), methotrexate, sulfasalazine, gold salts, azathioprine, cyclosporine, antimalarials, oral steroids (e.g., prednisone) or colchicine. Non-steroidal anti-inflammatories may also be coadministered with the IL-18 antagonist, including but not limited to: salicylic acid (aspirin); ibuprofen; indomethacin; celecoxib; rofecoxib; ketorolac; nambumetone; piroxicam; naproxen; oxaprozin; sulindac; ketoprofen; diclofenac; other COX-1 and/or COX-2 inhibitors, salicylic acid derivatives, propionic acid derivatives, acetic acid derivatives, fumaric acid derivatives, carboxylic acid derivatives, butyric acid derivatives, oxicams, pyrazoles and pyrazolones, including newly developed anti-inflammatories.

Moreover, the IL-18 antagonist can be used to treat psoriatic arthritis in combination with topical steroids, systemic steroids, antagonists of inflammatory cytokines, antibodies against T cell surface proteins, anthralin, coal tar, vitamin D3 and its analogs (including 1,25-dihydroxy vitamin D3 and calcipotriene), topical retinoids, oral retinoids (including but not limited to etretinate, acitretin and isotretinoin), topical salicylic acid, methotrexate, cyclosporine, hydroxyurea and sulfasalazine. In addition, it can be administered in combination with one or more of the following compounds; minocycline; misoprostol; oral collagen; penicillamine; 6-mercaptopurine; nitrogen mustard; gabapentin; bromocriptine; somatostatin; peptide T; anti-CD4 monoclonal antibody; fumaric acid; polyunsaturated ethyl ester lipids; zinc; and other drugs that can be used to treat psoriasis.

It is understood that the response by individual patients to the aforementioned medications or combination therapies may vary, and the most efficacious combination of drugs for each patient will be determined by his or her physician.

Further, in addition to human patients, IL-18 antagonists are useful in the treatment of non-human animals, such as pets (dogs, cats, birds, primates, etc.), domestic farm animals (horses cattle, sheep, pigs, birds, etc.), or any animal that suffers from an IL-18-mediated inflammatory or arthritic condition. In such instances, an appropriate dose may be determined according to the animal's body weight. For example, a dose of 0.2-1 mg/kg may be used. Alternatively, the dose is determined according to the animal's surface area, an exemplary dose ranging from 0.1-20 mg/m², or more preferably, from 5-12 mg/m². For small animals, such as dogs or cats, a suitable dose is 0.4 mg/kg. Soluble IL-18 receptor (preferably constructed from genes derived from the recipient species), or another soluble IL-18 antagonist, is administered by injection or other suitable route one or more times per week until the animal's condition is improved, or it may be administered indefinitely.

### Additional Diseases Treatable By IL-18 Antagonists

The disclosed experimental data demonstrates that IL-18 antagonists can be used to treat inflammatory conditions associated with IL-18. Accordingly, a number of other diseases are treatable with IL-18 antagonists.

Cardiovascular disorders are treatable and/or preventable with the disclosed IL-18 antagonists, pharmaceutical compositions or combination therapies. In particularly, cardiovascular disorders are treatable with IL-18 antagonist compositions, alone or in combination with TNF inhibitors (e.g. ENBREL) and or other agents as described above. Cardiovascular disorders thus treatable include aortic aneurysms; arteritis; vascular occlusion, including cerebral artery occlusion; complications of coronary by-pass surgery; ischemia/reperfusion injury; heart disease, including atherosclerotic heart disease, myocarditis, including chronic autoimmune myocarditis and viral myocarditis; heart failure, including chronic heart failure (CHF), cachexia of heart failure; myocardial infarction; restenosis and/or atherosclerosis after heart surgery or after carotid artery balloon angioplastic procedures; silent myocardial ischemia; post implantation complications of left ventricular assist devices; Raynaud's phenomena; thrombophlebitis; vasculitis, including Kawasaki's vasculitis; veno-occlusive disease, giant cell arteritis, Wegener's granulomatosis; mental confusion following cardio pulmonary by pass surgery, and Schoenlein-Henoch purpura. Combinations of IL-18 antagonists, TNF inhibitors and angiogenesis inhibitors (e.g. anti-VEGF) are useful for treating certain cardiovascular diseases such as aortic aneurysms and tumors.

In addition, the subject IL-18 antagonists, compositions and combination therapies are used to treat chronic pain conditions, such as chronic pelvic pain, including chronic prostatitis/pelvic pain syndrome. As a further example, soluble IL-18 receptor and the compositions and combination therapies of the invention are used to treat post-herpetic pain.

Provided also are methods for using IL-18 antagonists, compositions or combination therapies to treat various disorders of the endocrine system. For example, IL-18 binding protein compositions or other IL-18 antagonist compositions, with or without TNF inhibitors (ENBREL) or other active agents described above, are suitable for use to treat juvenile onset diabetes (includes autoimmune and insulin-dependent types of diabetes) and also to treat maturity onset diabetes (includes non-insulin dependent and obesity-mediated diabetes). In addition, the subject compounds, compositions and combination therapies are used to treat secondary conditions associated with diabetes, such as diabetic retinopathy, kidney transplant rejection in diabetic patients, obesity-mediated insulin resistance, and renal failure, which itself may be associated with proteinurea and hypertension. Other endocrine disorders also are treatable with these compounds, compositions or combination therapies, including polycystic ovarian disease, X-linked adrenoleukodystrophy, hypothyroidism and thyroiditis, including Hashimoto's thyroiditis (i.e., autoimmune thyroiditis). Further, IL-18 antagonists, including IL-18 receptor or IL-18 binding protein, alone or in combination with other cytokines, including TNF inhibitors such as ENBREL, are useful in treating or preventing medical conditions associated with thyroid cell dysfunction, including euthyroid sick syndrome.

Included also are methods for using the subject IL-18 antagonists, compositions or combination therapies for treating disorders of the genitourinary system. For example, IL-18 antagonist compositions, alone or in combination with TNF inhibitors (ENBREL) or other active agents described above are suitable for treating or preventing nephrotic syndrome and/or glomerulonephritis, including autoimmune glomerulonephritis, glomerulonephritis due to exposure to toxins or glomerulonephritis secondary to infections with haemolytic streptococci or other infectious agents. Also treatable with the compounds, compositions and combination therapies of the invention are uremic syndrome and its clinical complications (for example, renal failure, anemia, and hypertrophic cardiomyopathy), including uremic syndrome associated with exposure to environmental toxins, drugs or other causes and renal failure due to ischemia. IL-18 antagonists, particularly soluble IL-18 receptor or IL-18 binding protein or an antibody, alone or in combination with TNF inhibitors, particularly ENBREL, are useful in treating and preventing complications that arise from inflammation of the gallbladder wall that leads to alteration in absorptive function. Included in such complications are cholelithiasis (gallstones) and choliedocholithiasis (bile duct stones) and the recurrence of cholelithiasis and choliedocholithiasis. Further conditions treatable with the compounds, compositions and combination therapies of the invention are complications of hemodialysis; prostate conditions, including benign prostatic hypertrophy, nonbacterial prostatitis and chronic prostatitis; and complications of hemodialysis.

Also provided herein are methods for using IL-18 antagonists, compositions or combination therapies to treat various hematologic and oncologic disorders. For example, soluble IL-18 receptor or IL-18 binding protein or an antagonistic antibody, alone or in combination with a TNF inhibitor (ENBREL) or other active agents as described above, can be used to treat various forms of cancer, including acute myelogenous leukemia, chronic myelogenous leukemia leukemia, Epstein-Barr virus-positive nasopharyngeal carcinoma, glioma, colon, stomach, prostate, renal cell, cervical and ovarian cancers, lung cancer (SCLC and NSCLC), including cancer-associated cachexia, fatigue, asthenia, paraneoplastic syndrome of cachexia and hypercalcemia. Additional diseases treatable with the subject IL-18 antagonists, compositions or combination therapies are solid tumors, including sarcoma, osteosarcoma, and carcinoma, such as adenocarcinoma (for example, breast cancer) and squamous cell carcinoma. In addition, the subject compounds, compositions or combination therapies are useful for treating esophogeal cancer, gastric cancer, leukemia, including acute myelogenous leukemia, chronic myelogenous leukemia, myeloid leukemia, chronic or acute lymphoblastic leukemia and hairy cell leukemia. Other malignancies with invasive metastatic potential, including multiple myeloma, can be treated with the subject compounds, compositions and combination therapies, and particularly combination therapies that include soluble IL-18 receptor and soluble TNF receptor (ENBREL). In addition, the disclosed IL-18 antagonists, compositions and combination therapies can be used to treat anemias and hematologic disorders, including anemia of chronic disease, aplastic anemia, including Fanconi's aplastic anemia; idiopathic thrombocytopenic purpura (ITP); thrombotic thrombocytopenic purpura, myelodysplastic syndromes (including refractory anemia, refractory anemia with ringed sideroblasts, refractory anemia with excess blasts, refractory anemia with excess blasts in transformation); myelofibrosis/myeloid metaplasia; and sickle cell vasocclusive crisis.

Various lymphoproliferative disorders also are treatable with the disclosed IL-18 antagonists, compositions or combination therapies. IL-18 antagonist, alone or in combination with a TNF inhibitor, such as ENBREL, or other active agents are useful for treating or preventing autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sezary syndrome.

In addition, the subject IL-18 antagonists, compositions and combination therapies are used to treat hereditary conditions. In particular, IL-18 antagonist, alone or in combination with a TNF inhibitor such as ENBREL, is useful to treat diseases such as Gaucher's disease, Huntington's disease, linear IgA disease, and muscular dystrophy.

Other conditions treatable or preventable by the disclosed IL-18 antagonists, compositions and combination therapies include those resulting from injuries to the head or spinal cord including subdural hematoma due to trauma to the head. For example, soluble IL-18 receptor, alone or in combination with a TNF inhibitor such as ENBREL are useful in treating head injuries and spinal chord injuries. In connection with this therapy, the compositions and combinations described are suitable for preventing cranial neurologic damage and preventing and treating cervicogenic headache.

The disclosed IL-18 antagonists, compositions and combination therapies are further used to treat conditions of the liver. For example soluble IL-18 receptor, alone or in combination with a TNF inhibitor such as ENBREL or other active agents, can be used to treat hepatitis, including acute alcoholic hepatitis, acute drug-induced or viral hepatitis, hepatitis A, B and C, sclerosing cholangitis and inflammation of the liver due to unknown causes. The invention is particularly useful in treating hepatitis due to Hepatitis C virus. In connection with liver inflammation, IL-18 antagonists are further useful in treating hepatic sinusoid epithelium and biliary atresia.

In addition, the disclosed IL-18 antagonists, compositions and combination therapies are used to treat various disorders that involve hearing loss and that are associated with abnormal IL-18 expression. For example, soluble IL-18 receptor, alone or in combination with TNF inhibitors, can be used to treat or prevent cochlear nerve-associated hearing loss that is thought to result from an autoimmune process, i.e., autoimmune hearing loss. This condition currently is treated with steroids, methotrexate and/or cyclophosphamide. Also treatable or preventable with the disclosed IL-18 antagonists, compositions and combination therapies is Meniere's syndrome and Scholesteatoma, a middle ear disorder often associated with hearing loss.

In addition, the subject invention provides IL-18 antagonists, e.g. soluble IL-18 receptor or IL-18 binding protein or an antagonistic antibody, compositions and combination therapies (e.g. soluble IL-18 receptor and a TNF inhibitor such as ENBREL or other active agents) for the treatment of non-arthritic medical conditions of the bones and joints. This encompasses osteoarthritis and periodontitis resulting in tooth loosening or loss, and prosthesis loosening after joint replacement (generally associated with an inflammatory response to wear debris). This latter condition also is called “orthopedic implant osteolysis.” Another condition treatable with the compounds, compositions and combination therapies of the invention is temporal mandibular joint dysfunction (TMJ).

The following pulmonary disorders also can be treated or prevented with the disclosed IL-18 antagonists, compositions and combination therapies (e.g. IL-18 antagonist and a TNF inhibitor such as ENBREL or other active agents): adult respiratory distress syndrome (ARDS), acute respiratory distress syndrome and acute lung injury caused by a variety of conditions, including exposure to toxic chemicals, pancreatitis, trauma or other causes of inflammation. The disclosed compounds, compositions and combination therapies of the invention also are useful for treating broncho-pulmonary dysplasia (BPD); chronic obstructive pulmonary diseases (e.g. emphysema and chronic bronchitis), and chronic fibrotic lung disease of preterm infants. In addition, the compounds, compositions and combination therapies of the invention are used to treat occupational lung diseases, including asbestosis, coal worker's pneumoconiosis, silicosis or similar conditions associated with long-term exposure to fine particles. In other aspects of the invention, the disclosed compounds, compositions and combination therapies are used to treat pulmonary fibrosis, including idiopathic pulmonary fibrosis and radiation-induced pulmonary fibrosis; pulmonary sarcoidosis; and allergies, including allergic rhinitis, contact dermatitis, atopic dermatitis and asthma.

The IL-18 antagonists, e.g. soluble IL-18 receptor, compositions and combination therapies (e.g. an IL-18 antagonist as soluble IL-18 receptor in combination with ENBREL or other TNF inhibitor or active agent) of the invention are useful for treating or preventing primary amyloidosis. In addition, the secondary amyloidosis that is characteristic of various conditions also are treatable with IL-18 antagonists such as soluble IL-18 receptor, and the compositions and combination therapies described herein. Such conditions include: Alzheimer's disease, secondary reactive amyloidosis; Down's syndrome; and dialysis-associated amyloidosis. Also treatable with the compounds, compositions and combination therapies of the invention are inherited periodic fever syndromes, including familial Mediterranean fever, hyperimmunoglobulin D and periodic fever syndrome and TNF-receptor associated periodic syndromes (TRAPS).

Disorders involving the skin or mucous membranes also are treatable using the disclosed IL-18 antagonists, compositions or combination therapies, e.g. soluble IL-18 receptor and ENBREL. Such disorders include acantholytic diseases, including Darier's disease, keratosis follicularis and pemphigus vulgaris. Also treatable with the subject IL-18 antagonists, especially soluble IL-18 receptor, compositions and combination therapies are acne; acne rosacea; alopecia greata; aphthous stomatitis; bullous pemphigoid; burns; eczema; erythema, including erythema multiforme and erythema multiforme bullosum (Stevens-Johnson syndrome); inflammatory skin disease; lichen planus; linear IgA bullous disease (chronic bullous dermatosis of childhood); loss of skin elasticity; mucosal surface ulcers; neutrophilic dermatitis (Sweet's syndrome); dermatomyositis, pityriasis rubra pilaris; psoriasis; pyoderma gangrenosum; multicentric reticulohistiocytosis; and toxic epidermal necrolysis.

Disorders associated with transplantation also are treatable or preventable with the disclosed IL-18 antagonists, such as soluble IL-18 receptor, compositions or combination therapies, including compositions of soluble IL-18 receptor and ENBREL. Such disorders include graft-versus-host disease, and complications resulting from solid organ transplantation, such as heart, liver, skin, kidney, lung (lung transplant airway obliteration) or other transplants.

Ocular disorders also are treatable or preventable with the disclosed IL-18 antagonists, especially soluble IL-18 receptor, compositions or combination therapies, including rhegmatogenous retinal detachment, and inflammatory eye disease, including inflammatory eye disease associated with smoking and macular degeneration.

IL-18 antagonists such as soluble IL-18 receptor and the disclosed compositions and combination therapies also are useful for treating disorders that affect the female reproductive system. Examples include, but are not limited to, multiple implant failure/infertility; fetal loss syndrome or IV embryo loss (spontaneous abortion); preeclamptic pregnancies or eclampsia; endometriosis, chronic cervicitis, and pre-term labor.

In addition, the disclosed IL-18 antagonists, particularly soluble IL-18 receptor or IL-18 binding protein or an antagonistic antibody, compositions and combination therapies, such as combinations of IL-18 antagonist and ENBREL are useful for treating obesity, including to bring about a decrease in leptin formation. Also, the compounds, compositions and combination therapies of the invention are used to treat or prevent sciatica, symptoms of aging, severe drug reactions (for example, IL-2 toxicity or bleomycin-induced pneumopathy and fibrosis), or to suppress the inflammatory response prior, during or after the transfusion of allogeneic red blood cells in cardiac or other surgery, or in treating a traumatic injury to a limb or joint, such as traumatic knee injury. Various other medical disorders treatable with the disclosed IL-18 antagonists, compositions and combination therapies include; multiple sclerosis; Behcet's syndrome; Sjogren's syndrome; autoimmune hemolytic anemia; beta thalassemia; amyotrophic lateral sclerosis (Lou Gehrig's Disease); Parkinson's disease; and tenosynovitis of unknown cause, as well as various autoimmune disorders or diseases associated with hereditary deficiencies, including x-linked mental retardation.

The disclosed IL-18 antagonists, particularly soluble IL-18 receptor, compositions and combination therapies, e.g. soluble IL-18 receptor and ENBREL, are useful for treating the effects of neurotoxic neurotransmitters discharged during excitation of inflammation in the central nervous system and to inhibit or prevent the development of glial scars at sites of central nervous system injury. In connection with central nervous system medical conditions, IL-18 antagonists, alone or in combination with TNF inhibitors and particularly IL-18 antagonist and/or ENBREL are useful in treating temporal lobe epilepsy. Furthermore, the disclosed IL-18 antagonists, particularly soluble IL-18 receptor or soluble IL-18 binding protein or an antagonistic antibody, compositions and combination therapies, e.g. soluble IL-18 receptor and ENBREL, furthermore are useful for treating acute polyneuropathy; anorexia nervosa; Bell's palsy; chronic fatigue syndrome; transmissible dementia, including Creutzfeld-Jacob disease; demyelinating neuropathy; Guillain-Barre syndrome; vertebral disc disease; Gulf war syndrome; chronic inflammatory demyelinating polyneuropathy, myasthenia gravis; silent cerebral ischemia; sleep disorders, including narcolepsy and sleep apnea; chronic neuronal degeneration; and stroke, including cerebral ischemic diseases. Other diseases and medical conditions that can be treated or prevented by administering an IL-18 antagonist, such as soluble IL-18 receptor, alone or in combination with a herein described active agents, particularly a TNF inhibitor such as ENBREL, include anorexia and/or anorexic conditions, peritonitis, endotoxemia and septic shock, granuloma formation, heat stroke, Churg-Strauss syndrome, chronic inflammation following acute infections such as tuberculosis and leprosy, systemic sclerosis and hypertrophic scarring. In addition to IL-18 antagonists in combination with TNF inhibitors, IFN-alpha beta or gamma and/or IL-4 inhibitors are suitable for treating hypertrophic scarring.

Provided herein are methods of treating or preventing psoriatic lesions that involve administering to a human patient a therapeutically effective amount of an IL-18 antagonist. A preferred antagonist for this purpose is a soluble antagonist such as a soluble IL-18 receptor or IL-18 binding protein or an antagonistic antibody to IL-18 or a component of the IL-18 receptor. The treatment is effective against psoriatic lesions that occur in patients who have ordinary psoriasis or psoriatic arthritis. In addition, any of the combination therapies enumerated above are useful for the treatment of psoriasis.

Patients are defined as having ordinary psoriasis if they lack the more serious symptoms of psoriatic arthritis (e.g., distal interphalangeal joint DIP involvement, enthesopathy, spondylitis and dactylitis), but exhibit one of the following: 1) inflamed swollen skin lesions covered with silvery white scale (plaque psoriasis or psoriasis vulgaris); 2) small red dots appearing on the trunk, arms or legs (guttate psoriasis); 3) smooth inflamed lesions without scaling in the flexural surfaces of the skin (inverse psoriasis); 4) widespread reddening and exfoliation of fine scales, with or without itching and swelling (erythrodermic psoriasis); 5) blister-like lesions (pustular psoriasis); 6) elevated inflamed scalp lesions covered by silvery white scales (scalp psoriasis); 7) pitted fingernails, with or without yellowish discoloration, crumbling nails, or inflammation and detachment of the nail from the nail bed (nail psoriasis).

In treating ordinary psoriasis, an IL-18 antagonist is administered in an amount and for a time sufficient to induce an improvement in the patient's condition as measured according to any indicator that reflects the severity of the patient's psoriatic lesions. One or more such indicators may be assessed for determining whether the amount of IL-18 antagonist and duration of treatment is sufficient. In one preferred embodiment of the invention, the soluble IL-18 receptor is administered in an amount and for a time sufficient to induce an improvement over baseline in either the psoriasis area and severity index (PASI) or the Target Lesion Assessment Score. In another embodiment, both indicators are used. When PASI score is used as the indicator, treatment is regarded as sufficient when the patient exhibits an at least 50% improvement in his or her PASI score, or alternatively, when the patient exhibits an at least 75% improvement in PASI score. Using the Psoriasis Target Lesion Assessment Score to measure sufficiency of treatment involves determining for an individual psoriatic lesion whether improvement has occurred in one or more of the following, each of which is separately scored: plaque elevation; amount and degree of scaling or degree of erythema; and target lesion response to treatment. Psoriasis Target Lesion Assessment Score is determined by adding together the separate scores for all four of the aforementioned indicia, and determining the extent of improvement by comparing the baseline score the score after treatment has been administered.

IL-18 antagonists such as an IL-18 receptor or IL-18 binding protein also can be administered in combination with GM-CSF, IL-2 and inhibitors of protein kinase A type 1 to enhance T cell proliferation in HIV-infected patients who are receiving anti-retroviral therapy.

Although administration of IL-18 has been described as useful in fighting infections, many complications from infection arise as a result of an overactive or insufficiently controlled immune response. Thus, the disclosed IL-18 antagonists, compositions and combination therapies described herein are useful in medicines for treating bacterial, viral or protozoal infections, and complications resulting therefrom. One such disease is Mycoplasma pneumonia. In addition, provided herein is the use of soluble IL-18 antagonist compositions or combinations, particularly in combination with ENBREL to treat AIDS and related conditions, such as AIDS dementia complex, AIDS associated wasting, lipidistrophy due to antiretroviral therapy; CMV (cytomegalovirus) and Kaposi's sarcoma. Furthermore provided herein is the use of soluble IL-18 antagonist compositions or combinations for treating protozoal diseases, including malaria and schistosomiasis. Additionally provided is the use of an IL-18 antagonist to treat erythema nodosum leprosum; bacterial or viral meningitis; tuberculosis, including pulmonary tuberculosis; and pneumonitis secondary to a bacterial or viral infection. Provided also herein is the use of IL-18 antagonist compositions or combinations to prepare medicaments for treating louse-borne relapsing fevers, such as that caused by Borrelia recurrentis. IL-18 antagonist can also be used to prepare a medicament for treating conditions caused by Herpes viruses, such as herpetic stromal keratitis, corneal lesions; and virus-induced corneal disorders. In addition, IL-18 antagonist compositions and combinations can be used in treating human papillomavirus infections. IL-18 antagonist is used also to prepare medicaments to treat influenza infection. Further, IL-18 antagonist compositions and combinations can be used to treat sepsis due to microbial infection.

The invention having been described, the following examples are offered by way of illustration, and not limitation.

### Example

### Effect of Antagonizing IL-18 in a Mouse Model of Rheumatoid Arthritis

This experiment was designed to test effect of antagonizing IL-18 in a mouse model of rheumatoid arthritis, the well-known collagen-induced arthritis model. As an IL-18 antagonist, a fusion protein was made between the IL-18 binding protein and an Fc mutein. The amino acid sequence of the resulting protein, IL-18BP-Fc, is given in SEQ ID NO:5. This protein was transiently expressed in CV-1/EBNA cells following DEAE-Dextran transfection of an expression vector, and purified from the culture supernatant on a protein A column. Purity was assessed by PAGE at greater than 98%.

In each experiment, male DBA/1 mice were immunized with collagen on day −21 and were boosted on day 0. Mice were treated daily from days 0-14 with IP injections of antagonists or control proteins. The incidence and severity of arthritis was monitored in a blind fashion. Each paw is assigned a score from 0 to 4 as follows: 0=normal; 1=swelling in 1 to 3 digits; 2=mild swelling in ankles, forepaws, or more than 3 digits; 3=moderate swelling in multiple joints; 4=severe swelling with loss of function. Each paw is totaled for a cumulative score/mouse. Then, cumulative scores are totaled for mice in each group for a mean clinical score.

In the first & second experiments, mice were treated with 150 μg/day of IL-18BP-Fc (n=10 in each experiment) and compared to mice treated with 150 μg/day of either entanercept (ENBREL®) (huTNFRFc) or Hu IgG (n=15/group in each experiment). In both experiments, mice treated with IL-18BP-Fc demonstrated a statistically significant reduction in the incidence and severity of the disease (73% reduction in mean clinical score in the first experiment, and 88% reduction in mean clinical score in the second experiment) compared with controls. ENBREL® treatment was also very effective at inhibiting the disease (92% reduction in mean clinical score in the first experiment, and 90% reduction in mean clinical score in the second experiment) compared with controls.

### Example

### Dose Response Experiment in a Mouse Model of Rheumatoid Arthritis

A third experiment in the same mouse model of CIA tested a dose response in which IL-18BP-FC was administered IP at 150, 50, 15, and 5 μg/day. As in the previous experiment, male DBA/1 mice were immunized with collagen on day −21 and were boosted on day 0. Mice were treated daily from days 0-14 with IP injections of antagonists or control proteins. The incidence and severity of arthritis was monitored in a blind fashion.

Groups of 15 mice were treated with the indicated doses of IL-18BP-Fc or with 150 μg/day of ENBREL® or Hu IgG. All doses of IL-18BP-Fc tested significantly reduced the disease incidence and severity of arthritis (63%-76% reduction in mean clinical score for IL-18BP-Fc treated mice; 85% reduction in mean clinical score for the huTNFRFc treated group) compared with the control group.

The results indicate that at each dose level, IL-18BP-Fc effectively inhibited onset of CIA when the reagent is administered in a preventative protocol. Thus, the minimally effective dose of IL-18BP-Fc in the preventative protocol could not be deduced using this range of dosages; an additional titration experiment is therefore done to determine the minimally effective dosage.

### Example

### Combination Treatment of an IL-18 Antagonist with Entanercept (ENBREL®)

The minimally effective dose of an IL-18 antagonist, in this case, IL-18BP-Fc, is co-administered along with minimally effective doses of entanercept (ENBREL®) to mice with CIA. Induction of arthritis and controls are as described above. The combination of IL-18BP-Fc and entanercept (ENBREL®) effectively inhibits CIA.

### Example

### Effect of Antagonizing IL-18 in a Therapeutic Mouse Model of Rheumatoid Arthritis

This experiment was designed to assess the effect of inhibiting IL-18 as a therapeutic agent after arthritis had been established. Thus, the protocol for initiation of arthritis via injections of collagen was the same as for the above experiments, except that treatment was not begun until after the mice had established disease symptoms. In this experiment, this point occurred at day 7 after the boost (28 days after the first treatment with collagen). Treatment with the following polypeptides at the indicated dose (daily injections, ip) was then performed for 13 days. Each treatment group consisted of 8 mice exhibiting signs of the disease. As before, an irrelevant human IgG was used as a negative control, and entanercept (ENBREL®) was used as a positive control. In addition, a monoclonal antibody to the IL-1R (M147) was also used as a positive control (Rogers et al, 1992, Proc. Natl. Acad. Sci. USA 89:1011-1015).

TABLE 1

| Treatment | Dose | Score after 13 days treatment |
|---|---|---|
| IL-18BP-Fc | 300 ug | 6.1 |
| HuIgG | 300 ug | 10.1 |
| Enbrel | 150 ug | 6.7 |
| anti-IL1R Ab (M147) | 50 ug | 0 |

These results demonstrated a beneficial effect of using an IL-18 antagonist as a therapeutic agent after arthritis had been established. Administration of IL-18BP-Fc did prevent progression of disease symptoms, in a manner similar to the results seen using ENBREL® in this animal model system.

### Example

### Antagonism of IL-18 in Two Different Mouse Models of Inflammatory Bowel Disease (IBD) with p13Fc

This experiment was designed to determine whether IL-18 plays a significant role in the pathology of IBD and, if so, can its effect be blocked in vivo with an IL-18 antagonist. The effect of blocking IL-18 was analyzed in both an experimentally induced model of IBD (the DSS model), and a spontaneous mouse model of IBD.

One of the most widely used models of IBD is the DSS model (dextran sulphate salt). In this model, dextran sulphate salt (m.w. can be 40,000 to 500,000, but usually use that around 40,000) is given to mice (or other small mammals) in their drinking water at 2% to 8%.

For the spontaneous mouse model of IBD, Mdr1 a knockout mice were used. These mice, which are homozygous for a deletion of function in the Mdr1 a locus, spontaneously develop inflammatory bowel disease.

In this experiment, p 13Fc was used as the IL-18 antagonist. p13Fc is an Fc fusion derivative of a fowlpox viral protein that binds to IL-18 (Born et al., 2000, J. Immunol. 164(6):3246-54).

### Mice and Experimental Design:

Two experimental groups of mice were studied: C57B1/6 mice with DSS-induced colitis, and Mdr1a knockout mice (Mdr1 a−/−) that develop spontaneous colitis. An irrelevant human IgG (HuIg) was used as a negative control, and a monoclonal antibody M147 previously shown to decrease weight loss in DSS-induced IBD was used as a positive control. The experimental groups were set up as follows:

C57BL/6 mice; n=4 mice per group

No DSS+HuIg (150 μg/day/mouse D0-D8)

3% DSS+p13Fc (150 μg/day/mouse D0-D8)

3% DSS+HuIgG (150 μg/day/mouse D0-D8)

3% DSS+M147 (250 μg/day/mouse D0-D8)

FVB and Mdr1a−/− mice; n=4 per group

Healthy FVB mice+HuIgG (150 μg/day/mouse D0-D10)

Sick Mdr1a−/− mice+p13Fc (150 μg/day/mouse D0-D10)

Sick Mdr1a−/− mice+HuIgG (150 μg/day/mouse D0-D10)

Sick Mdr1a−/− mice+M147 mAb (250 μg/day/mouse D0-D10)

Mice were weighed daily; weight loss is a clinical sign of the disease. Tissues were harvested at day 8 (D8) in the DSS model experiment, and at day 19 (D10) in the Mdr1a−/− model experiment. Histopathology (2 mice) was performed on the following tissues: small intestine, large intestine and mesenteric lymph nodes (MLN).

### Results and Conclusion

Preliminary histological analysis indicates that in the DSS model, inflammation in the large intestine was mildly reduced. The results from these preliminary experiments demonstrated that p13Fc can attenuate some of the weight loss induced by DSS-induced colitis, and some of the weight loss associated with ongoing exacerbation of spontaneous colitis.

### Example

### Antagonism of IL-18 in the DSS Mouse Model of Inflammatory Bowel Disease (IBD) With IL-18BP

This experiment was designed to analyze the effect of blocking IL-18 with IL-18BP-Fc, another IL-18 antagonist, during experimentally induced IBD. The DSS model of IBD was used. Controls were as described in the previous experiment. The sequence of the IL-18BP-Fc protein used is given in SEQ ID NO:5.

### Mice and Experimental Design:

C57BL/6 mice were given 2% DSS from day 0 to day 7 (D0-7); n=4 mice per group Groups:

No DSS+huIgG (250 μg/day/mouse D0-D7)

2% DSS+IL-18BP-Fc (60 μg/day/mouse D0-D7)

2% DSS+IL-18BP-Fc (100 μg/day/mouse D0-D7)

2% DSS+huIgG (250 μg/day/mouse D0-D7)

2% DSS+M147 (250 μg/day/mouse D0-D7)

Mice were weighed daily. Tissues (intestine and MLN) were harvested at day 8 (D8). Histopathology (2 mice) analyses were also performed on the tissues.

### Results and Conclusion

Preliminary histological analysis indicated that inflammation in the large intestine was mildly reduced. The results from these experiments also showed that IL-18BP-Fc is able to attenuate some of the weight loss associated with DSS-induced colitis.

### Example

### Dose Response Experiments Using IL-18BP-Fc in the DSS Mouse Model of Inflammatory Bowel Disease (IBD)

In order to further examine the effect of antagonizing IL-18 in a mouse model of IBD, IL-18BP-Fc was administered at a higher range of dosages.

### Mice and Experimental Design

C57BL/6 mice were given 2% DSS in their drinking water for 7 days (D0-7). Treatment groups were as below; each treatment group contained 8 mice.

No DSS+huIgG (150 μg/day/mouse)
DSS+IL-18BP-Fc (300 μg/day/mouse)
DSS+IL-18BP-Fc (100 μg/day/mouse)
DSS+huIgG (150 μg/day/mouse)
DSS+M147 (250 μg/day/mouse)

Mice were analyzed for weight loss daily. At the end of the experiment, large intestine, small intestine and MLN were taken for histology and for RNA analysis by RNase protection assay. MLN cells were also counted, and analyzed by flow cytometry (FACs) for cell phenotyping and for cytokine production in vitro by ELISA (after stimulation with anti-CD3).

### Results and Conclusions

The higher dose of IL-18BP-Fc (300 μg) inhibited weight loss by about 32-35% on Day 8 of treatment. IL-18BP-Fc also inhibited the increased cellularity (increased number of total cells per MLN) that is typically seen in the MLN after DSS treatment, suggesting that it was blocking the process of cellular infiltration during inflammation.

Cytokine production by MLN cells after stimulation with CD3 was examined. This analysis indicated that IL-18BP-Fc inhibited the increased IFNγ production that would otherwise occur during DSS-induced colitis. Histological data also showed decreased inflammation in the large intestine after administration of IL-18BP-Fc at the higher dose.

Using RNase Protection Analysis, it was also observed that IL-18BP-Fc decreased the levels of RNA encoding IL-1α/β and the IL-1 receptor antagonist (IL-1RA) in the DSS model. This result is indicative of reduced IL-1 production and possibly reduced inflammation in the gut.

### Example

### Effect of Increased Dosage of IL-18BP-Fc in the DSS Mouse Model of Inflammatory Bowel Disease (IBD)

One possibility for the moderate effect of IL-18BP-Fc that was observed in the previous experiment was that the dose was not optimal. Accordingly, another experiment was performed with a higher dose of IL-18BP-Fc (600 μg/mouse/day).

### Mice and Experimental Design

| Mice | #/group 2% | DSS treatment | (D −2--> D7) |
|---|---|---|---|
| C57B1/6 | 6 | No | None |
| C57BL/6 | 6 | Yes | 250 μg/day HuIgG |
| C57B1/6 | 6 | Yes | 600 μg/day IL-18BP-Fc |
| C57BL/6 | 6 | Yes | 250 μg/day M147 |

Mice were analyzed for weight loss daily. At the end of the experiment, large intestine, small intestine and MLN were taken for histology and for RNA analysis. The number of cells in each MLN was counted (by dilution and staining of a sample) and an average for each group determined. In addition, the MLN cells were analyzed by flow cytometry (FACs) for cell phenotyping and for cytokine production in vitro by ELISA (after stimulation with anti-CD3).

### Results and Conclusions

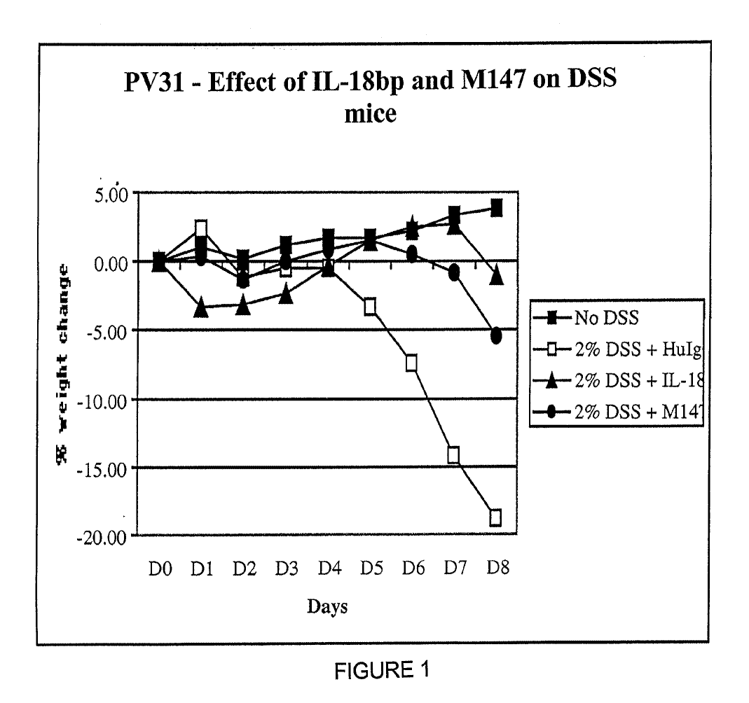

The effect of treatment on weight loss over the 8 day course of the experiment is illustrated in FIG. 1. Both M147 and IL-18BP-Fc significantly inhibited weight loss (85-90%) in the DSS-induced colitis model. Histological analysis indicated that there was reduced inflammation in the large intestine in the IL-18BP-Fc-treated and M 147-treated groups, as compared to mice treated with a control antibody (human IgG).

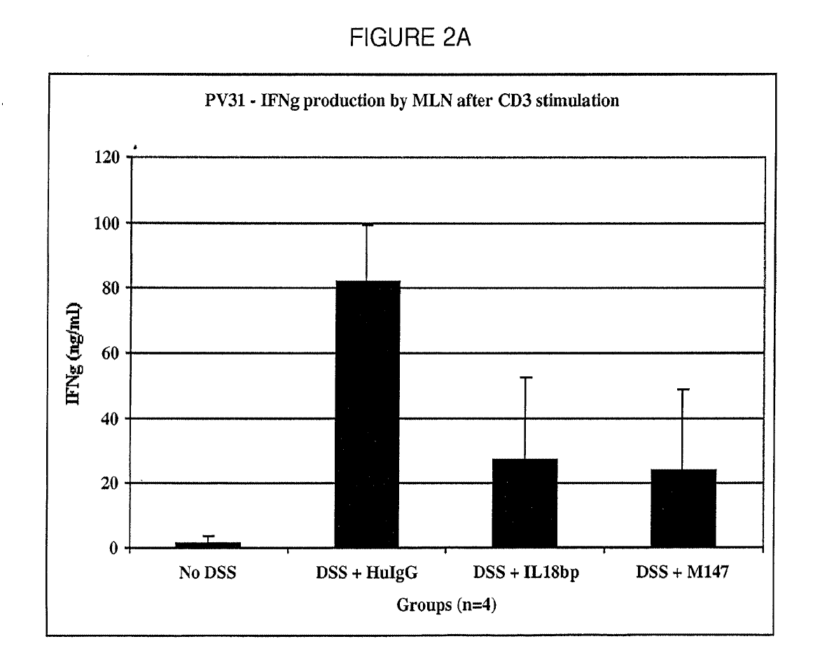

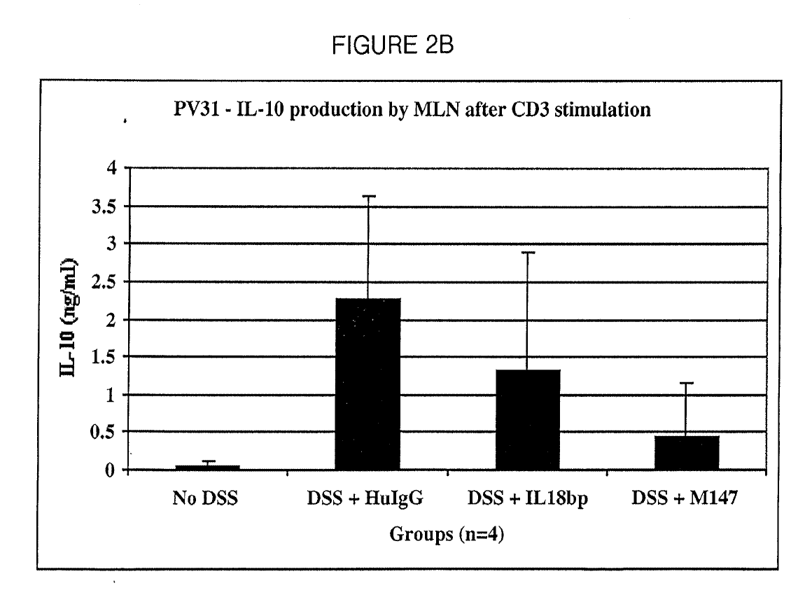

DSS treatment increases the levels of MLN cellularity (average number of cells per MLN per treatment group) by about 2-fold. MLN cellularity was decreased to control levels (that seen with no DSS treatment) in IL-18BP-Fc-treated and M147-treated mice. The MLN cells from DSS-treated mice, when stimulated by CD3, drastically increase IFNγ and IL-10 production as compared to control (no DSS) treated mice. Administration of either IL-18BP-Fc or M147 attenuated significantly this response (see FIG. 2A, which illustrates the average level of IFNγ production from each treatment group, and FIG. 2B, which illustrates the average level of IL-10 production).

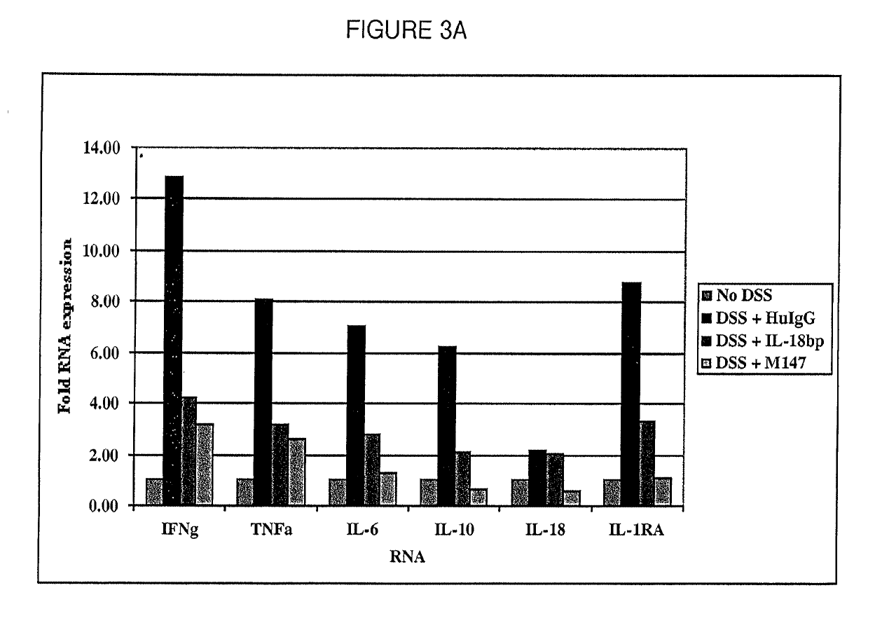

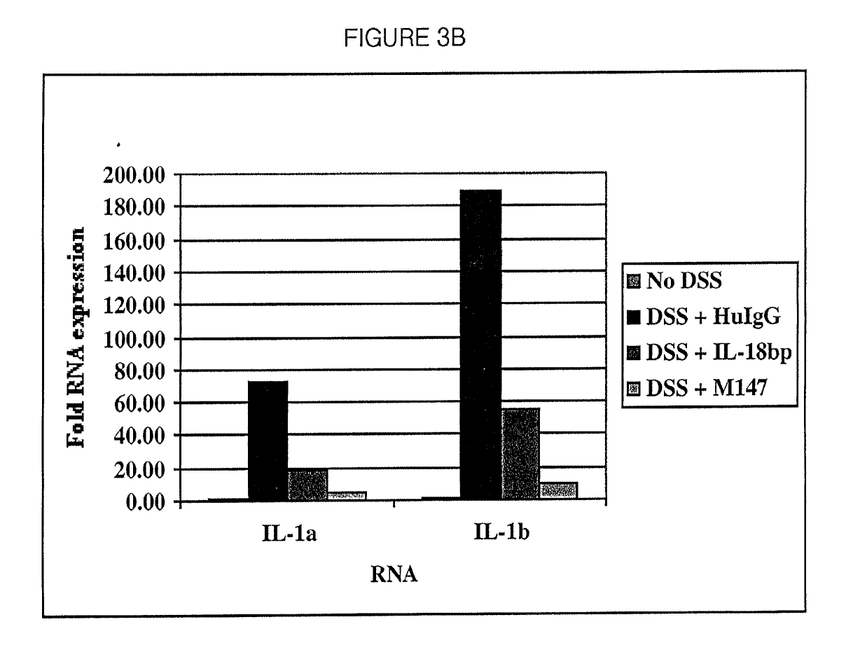

RNA analysis using both RNase Protection Assays as well as DNA arrays showed reduced levels of the mRNAs encoding multiple different indicators of inflammation. These indicators included inflammatory cytokines such as IL-1α/β, TNFα and IFNγ, as well as proteins involved in tissue repair including matrix metalloproteinases (MMPs). The results of RNase Protection Assays (RPA) detecting mRNAs in the large intestine encoding for IFNγ, TNFα, IL-6, IL-10, IL-18 and IL-1RA are shown in FIG. 3A. The results from an RNase Protection Assay (RPA) detecting mRNAs in the large intestine encoding for IL-1α and IL-1β are shown in FIG. 3B. These results show that upregulation of inflammatory genes by DSS treatment was attenuated when IL-18BP-Fc or M147 are administered. IL-18 RNA was however not regulated after IL-18BP-Fc treatment indicating that transcription of IL-18 was not affected in IL-18BP-Fc treated mice, and suggesting that IL-18BP-Fc does not regulate IL-18 by a transcriptional feedback mechanism. In conclusion, administration of IL-18BP-Fc clearly attenuated the weight loss and inflammation associated with DSS-induced colitis in mice.

### Example

### Effect of IL-18BP-Fc in the DSS Mouse Model of Inflammatory Bowel Disease (IBD) on MLN Cellularity and Chemokine Secretion

One of the major characteristics of inflammation in the gastrointestinal tract is that the cellularity of the MLN, the major lymph node draining from the gut, increases compared with non-colitic mice. This change in cellularity is due to infiltrating mononuclear cells, such as T cells and macrophages. Analysis of T cell populations and numbers in the MLN revealed an increase in the relative proportion and absolute number of CD8⁺ T cells compared with CD4⁺ T cells. IL-18BP-Fc inhibited the increased cellularity seen in MLN of mice with DSS-colitis and analysis of the T cell populations and CD4⁺/CD8⁺ T cell ratios showed that the numbers and the ratios of T cells in the MLN from IL-18BP-Fc-treated mice are similar to non-DSS treated groups.

Because cytokines have been shown to play an important role in multiple models of colitis and as we observed changes in cytokine mRNA profiles in the intestine, we were interested in determining whether cytokine protein profiles of cells draining from the gut would be modulated by IL-18 bp treatment. We analyzed MLN from the various groups of animals on d8 for the in vitro secretion of IL-4, IL-10 and IFN-alpha. MLN cells were cultured on either PBS or anti-CD3 coated plates for 48 hours and the culture supernatants analyzed by ELISA for cytokines. MLN taken from mice with DSS colitis showed increased levels of both IFN-alpha and IL-10 protein production following anti-CD3 treatment. In contrast, MLN from IL-18BP-Fc treated DSS colitic animals did not show the same increase in the levels of IFN-alpha and IL-10 protein. Although the protein levels were not identical to control non-DSS levels, the decrease for IFN-alpha was significant (p=0.003). IL-4 was below detectable levels for all samples in these experiments (data not shown). These results show that treatment with IL-18 bp inhibits the process of inflammation during DSS-induced colitis presumably by attenuating the trafficking of T cells into the MLN and thus attenuating the increased cytokine secretion in the gut associated lymphoid tissues.

### Example

### Analysis of Chemokine/Chemokine Receptor and MMP Gene Regulation in IL-18BP-Fc Treated Mice During DSS Colitis

To further characterize an expanded set of genes, RNA from the LI of the animals from the various treatment groups (d8) were used in array analysis using Affymetrix chips. Approximately 300 genes showed greater than 3-fold regulation after DSS treatment and counter regulation following IL-18BP-Fc treatment.

Focussing on chemokine and chemokine receptor gene regulation, we observed increases in MIP1-alpha, MIP1-beta, MIP2, RANTES, CCR2, and CCR5 during DSS colitis. Treatment with IL-18BP-Fc attenuated the upregulation of these genes indicating that IL-18 bp treatment may act upstream and be able to block the initiation of the chemokine inflammatory cascade associated with colitis. We also saw upregulation of ENA-78 (24×) and MIG (20×) in the LI of DSS colitic mice. IL-18BP-Fc treatment downregulated expression of these genes. These data are consistent with our histopathological analysis shown in Table I that indicates less recruitment of cells to the mucosa in IL-18 bp treated mice.

With regards to tissue repair and remodeling mechanisms during inflammation, a number of investigators have documented an increase in expression of a number of MMPs in human IBD including stromelysins (MMP-3 and 10), gelatinase B (MMP-9), collagenases (MMP-1, 8 and 13) and type IV collagen, as well as increases in tissue inhibitor of metalloproteinase (TIMP-1). Here we report an increase in RNA levels for MMP-3, 7, 9, 10, 13 and TIMP-1 in the LI of mice treated with DSS. Treatment with IL-18BP-Fc decreases mRNA levels for these MMPs and TIMP-1, down to that seen in control tissue, again indicating that blocking IL-18 attenuates the damage incurred during the initiating inflammatory stages of IBD.

In summary, these examples clearly demonstrate that blocking IL-18 function in vivo is an effective method of attenuating intestinal inflammation induced by DSS. These data indicate a role for the IL-18 pathway in the initiation of intestinal damage associated with IBD, that that IL-18 antagonists can be used as a therapeutic approach for treating IBD in humans.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. A method of treating a patient afflicted with a pulmonary disorder, the method comprising administering to said patient a therapeutically effective amount of an IL-18 antagonist.

2. The method of claim 1, wherein the IL-18 antagonist is soluble IL-18 receptor.

3. The method of claim 2, wherein the soluble IL-18 receptor is a heterodimeric receptor.

4. The method of claim 1, wherein the IL-18 antagonist is an antibody.

5. The method of claim 4, wherein the antibody immunospecifically recognizes a component of an IL-18 receptor.

6. The method of claim 4, wherein the antibody is a humanized antibody.

7. The method of claim 6, wherein the antibody is a single-chain antibody.

8. The method of claim 1, wherein the IL-18 antagonist is a soluble IL-18 binding protein.

9. The method of claim 1, wherein the IL-18 antagonist is administered one or more times per week.

10. The method of claim 1, wherein the IL-18 antagonist is administered by subcutaneous injection.

11. The method of claim 1, wherein the pulmonary disorder is selected from the group consisting of broncho-pulmonary dysplasia (BPD); chronic obstructive pulmonary diseases (COPD), and chronic fibrotic lung disease of preterm infants.

12. The method of claim 11, wherein the pulmonary disorder is COPD.

13. The method of claim 12 wherein the COPD is emphysema.

14. The method of claim 12, wherein the COPD is chronic bronchitis.

15. The method of claim 12 wherein the IL-18 antagonist is an antibody.

16. The method of claim 15, wherein the antibody immunospecifically recognizes a component of an IL-18 receptor.
